(19) United States
(12) Patent Application Publication
(10) Pub. No.: US 2006/0121548 A1
(43) Pub. Date: Jun. 8, 2006

(54) Systems and methods for measuring sodium concentration in saliva

(75) Inventors: David J. Robbins, Stevenson Ranch, CA (US); Charles G. Hwang, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Remote Clinical Solutions, Inc., San Francisco, CA (US)

(21) Appl. No.: 11/270,194

(22) Filed: Nov. 8, 2005

Related U.S. Application Data
(60) Provisional application No. 60/626,676, filed on Nov. 9, 2004.

(51) Int. Cl. C12Q 1/34; A61M 35/00; C12M 1/34

(52) U.S. Cl. 435/18; 435/287.1; 604/1

(57) ABSTRACT

Systems and methods for measuring saliva sodium concentration using a chromatographic reaction enable rapid-results, low-cost diagnosis of various medical conditions in an outpatient setting. In one embodiment, measured patient saliva sodium concentration is used by the patient or the patient's healthcare provider to guide medical decision making. In another embodiment, measured patient saliva sodium concentration is processed to mechanically adjust the concentration of sodium in an aqueous solution to be delivered to the patient for oral administration. In yet another embodiment, a closed loop system measures saliva sodium concentration and uses any of a number of different types of feedback control systems to monitor and control the fluid and/or electrolyte state of the patient.

### CROSS-REFERENCES TO RELATED APPLICATIONS

The present application is a non-provisional of U.S. Patent Application Ser. No. 60/626,676 (Attorney Docket No. 022337-000300US), filed Nov. 9, 2004, which is related to that of co-pending provisional application No. 60/603,949 (Attorney Docket No. 022337-000200US), filed on Aug. 23, 2004, the full disclosures of which are incorporated herein by reference.

### BACKGROUND OF THE INVENTION

1. Field of the Invention

The subject matter of This application relates to methods and systems for determining the concentration of sodium in saliva.

Certain populations are particularly at risk for developing various fluid and electrolyte disorders-among them, hypernatremia (elevated blood sodium levels), hyponatremia (depleted blood sodium levels), volume depletion, and edema—including independent seniors (for whom dehydration ranks among the top five most frequent causes for hospitalization), institutionalized seniors (of whom over 50 percent acquire hypo- or hypernatremia in a given 12-month period), young children (for whom dehydration resulting from gastroenteritis accounts for 10 percent of pediatric hospital admissions), post-surgical hospital patients (of whom between 5 percent and 15 percent develop hyper- or hyponatremia), professional and non-professional athletes (for whom dehydration of as little as 2 percent (dehydration of between 5 and 10 percent is common) can reduce athletic performance by as much as 20 percent), chronically-ill individuals (a number of chronic conditions, or medications for such conditions, precipitate dehydration, including diabetes and hypertension), military personnel, and mining and forestry personnel. Dehydration can lead to a number of serious medical complications, including renal failure, heart failure, brain damage, heat stroke, and death. If not treated in a timely fashion, mortality rates may exceed 50 percent. In 2000, the costs associated with dehydration-related hospitalizations among the 65+demographic alone totaled $3.8 billion.

Dehydration, or risk thereof, is extraordinarily difficult to monitor. First, severe dehydration can occur very rapidly, in just a couple of hours. Second, many of the symptoms associated with dehydration (e.g. fatigue, confusion, dry mouth) do not appear until substantial fluids have been lost and medical complications take hold. Finally, many of the symptoms of dehydration may be present in normally-hydrated, at-risk individuals (among seniors, for example, a number of chronic conditions, and medications for such conditions, cause confusion; among athletes, anaerobic exercise often causes dry mouth and/or fatigue). The implication of the latter is that individuals at risk for dehydration, or their health care providers, often attribute classic signs of dehydration to other conditions and do not seek to correct the condition as a result.

Provided some sufficient amount of water is consumed, sodium replacement is the most important factor in achieving, and maintaining, effective fluid balance. While the total volume of fluids lost (via sweat, for example) is often recommended as a guide for fluid replacement, it is generally understood that the latter is not the primary determinant of fluid retention. Clinical studies indicate that athletes retain only 37% of a low-sodium fluid (versus 71% of a high-sodium fluid). This means that athletes consuming a volume equal to twice their sweat loss do not achieve positive fluid balance when drinking a low-sodium beverage.

Sodium replacement requirements vary dramatically across patient populations, and among individuals over time, based on a number of different environmental, physical, and behavioral factors-including heat, humidity, altitude, sweat rate, cardiovascular fitness, diet, alcohol and caffeine consumption, type or management of acute or chronic conditions, and genetic variations. In fact, the National Athletic Trainers' Association defines the optimal oral rehydration solution as containing between 70 mg and 1266 mg of sodium per an 8 oz. solution. That is, the standard deviation around the mean sodium replacement requirement is high.

Correcting fluid and electrolyte disorders is extraordinarily difficult. Because sodium replacement requirements are unknown, individuals are left to formulate their own “best-guess” estimates of fluid and electrolyte replacement needs. These best-guess estimates are rarely accurate, as the deaths of Orioles pitcher Steve Bechler (2003), Minnesota Vikings offensive tackle Korey Stringer (2001), marathoners Rachel Townsend (2003), Cynthia Lucero (2002), and Kelly Barrett (1998), and a number of military trainees, among many others, bear testimony to.

The field of hydration monitoring and rehydration therapy is active. Its importance lies in facilitating early detection and correction. Ideally, at-risk patients, or their healthcare providers, would be able to frequently, inexpensively, and non-invasively measure sodium replacement requirements and adjust rehydration therapy accordingly, thereby keeping serum fluid and electrolyte levels close to normal physiological levels. Such a system would reduce medical complications, improve athletic performance, and provide obvious increases in quality of life for at-risk patients.

It is known that information derived from biometric data, for example analyte levels in body fluids, may be employed to reliably predict the onset of, or to indicate the presence of, a fluid or electrolyte disorder in a human patient. For example, for patients presenting symptoms of fluid or electrolyte disorders, physicians will often order lab tests which measure any of a number of different clinical parameters in body fluids-most often in blood or urine-including: sodium concentrations, osmolality, blood urea nitrogen (BUN) levels, creatinine levels, BUN/creatinine ratios, hematocrit levels, protein levels, glucose levels, keytone levels, amylase levels, calcium levels, urate levels, chloride levels, albumin levels, and urine specific gravity. Other non-analyte measures used to improve the accuracy of diagnosis and to guide rehydration therapy include weight change, mucous membrate moistness, reported renal function, urine volume, urine color, tissue turgor, venous pressure, postural change in heart rate, postural change in blood pressure, body temperature, respiratory rate, heat rate, blood pressure, medication and medical history, recent environmental conditions (e.g. heat, exercise, etc.), recent change in functional ability (e.g. cognitive function, continence, etc.), fever/diarrhea/vomiting, and recent fluid intake. Serum osmolality and serum sodium concentration are considered the gold standard tests.

A major drawback of such tests is that: 1) they must be performed in a hospital setting (patients operating in an outpatient setting cannot monitor fluid balance and adjust rehydration therapy accordingly), 2) they are often invasive, 3) technicians specifically trained in blood handling are often required to perform the tests, 4) the tests must often be sent to a lab for processing (e.g. expensive lab equipment is required), and 5) time-to-test-completion is slow.

As a diagnostic fluid, saliva offers distinct advantages over serum. Saliva can be collected rapidly and non-invasively, with little training, at a fraction of the cost of blood, in an outpatient environment.

Clinical studies demonstrate strong correlations (mean r=0.94, P<0.01) between saliva osmolality and hydration status including, among others, a recent study conducted by the School of Sport, Health and Exercise Sciences at the University of Wales (“Saliva flow rate, total protein concentration and osmolality as potential markers of whole body hydration status during progressive acute dehydration in humans,” Archives of Oral Biology (2004) 49, 148-154).

It is generally understood that serum osmolality is primarily a function of serum sodium concentration. And clinical studies show direct correlations between serum osmolality and serum sodium concentration, including a recent study conducted by Doctors Alexander Kratz, M.D., Ph.D., M.P.H., Elizabeth Lee-Lewandrowski, Ph.D., M.P.H., and Kent B. Lewandrowski, M.D. from the Division of Laboratory Medicine, Department of Pathology, Massachusetts General Hospital and Harvard Medical School; Dr. Arthur Siegel, M.D. from the Department of Medicine, McLean Hospital, Belmont, Massachusetts and Harvard Medical School; Dr. Joseph Verbalis, M.D. from Georgetown University Hospital; Dr. Marvin Adner, M.D. from Metrowest Medical Center; and Dr. Terry Shirey, Ph.D. from Nova Biomedical Corporation (see FIG. 5).

Thus, there exists a need for a disposable, low-cost, non-invasive, rapid-results system that measures saliva sodium concentration, a marker for dehydration as well as a number of other medical conditions.

2. Description of the Background Art

The sodium (Na+) effect on the activity of beta-galactosidase in the presence of other cations such as K+ and Mg²⁺ has been studied since at least 1950. Cohn and Monod (1951) investigated the action of various ions for enzymatic hydrolysis of lactose. The activity of monovalent cations was found to be complex. Depending on conditions such as the presence of certain other cations, Na+ can behave either as an inhibitor or as an activator. Monod et al. (1951) extended this work by investigating the effects of Na+ and K+ on beta-galactosidase inhibition by melibiose (an alpha-galactosidase).

Lederberg (1950) found that Na+ was conducive to the maximum rates of o-nitrophenyl beta-D-galactosidase (oNPG) hydrolysis. Kuby and Lardy (1953), however, stated that the effect of Na+ was nonvariant with substrate type, while Cohn and Monod (1951) found K+ promoted a greater hydrolysis rate when lactose was the substrate. The work of Reithel and Kim attempted to reconsider the monovalent cation effects of beta-galactosidase activity based on the hypothesis that previous studies were performed with non-homogeneous preparations of E. coli. They found that K+ is the most effective stimulator if lactose is the substrate. If oNPG is the substrate, then Na+ and Mg²⁺ must be present to obtain the maximum catalysis rate.

Becker and Evans (1969) found that Na+ affinity for beta-galactosidase was greater than that of K+ for oNPG and p-nitrophenyl Galactopyranoside (pNPG) and lactose. The activity of pNPG hydrolysis by K+ was inhibited by Na+. The activity of oNPG hydrolysis by Na+ was stimulated by K⁺. They concluded that the mechanism of Na+-mediated hydrolysis is different from the mechanism of K+ hydrolysis. Finally, Hill and Huber (1971) showed that beta-galactosidase can be inhibited by high concentrations of ions. This effect is reversible upon dilution. The Na+ activity profile has a broad peak for a given Mg²⁺ concentration.

Numerous patents have issued concerning the measurement of sodium concentration, and the use of beta-galactosidase for this and other purposes. U.S. Pat. No. 4,649,123 describes a test means for determining the presence of an ion in an aqueous test sample, the test means comprising a hydrophilic carrier matrix incorporated with finely divided globules of a hydrophobic vehicle, said vehicle containing an ionophore capable of forming a complex with a specific ion to be determined, and a reporter substance capable of interacting with the complex of the ionophore and the ion to produce a detectable response. A continuation patent—U.S. Pat. No. 5,300,439—applies the technology to a test pad device. Specific ionophores, reporter labels, and hydrophilic polymers are listed. The test pad includes a chelator. This patent extends the use of the ionophore chemical reaction or binding events to a potential hand-held device. In contrast to the present invention, detection is accomplished via a hydrophobic reporter substance such as a phenol, an indophenol compound, a triphenylmethane, a fluorescein, a fluorescein ester, a 7-hydroxy coumarin, a resorufin, a pyren-3-ol, or a flavone (rather than via an enzymatic reaction), and the chemicals involved remain toxic and ill suited for oral contact.

Similarly, U.S. Pat. No. 4,812,400 provides for a process for measuring the sodium concentration of a biological fluid, comprising the steps of supplying predetermined amounts of adenosine-5′-triphosphate (ATP), adenosine triphosphatase (ATPase), magnesium, and potassium in the presence of a buffer in a reaction mixture. In contrast to the present invention, the described assay uses the ATPase enzyme, rather than the beta-galactosidase enzyme, and measures a purple color vs. a standard curve. The described assay thus requires a spectrophotometer or other quantitating instrument, and is designed for a laboratory environment.

U.S. Pat. No. 5,700,652 provides for a method for quantitative determination of sodium by reacting the sample with beta-galactosidase in the presence of potassium, cesium, and/or ammonium ions. The reaction occurs in the presence of a crown ether. The use of beta-galactosidase for correlation of reaction result with sodium content has been known since at least 1971 (Hill, BBA 250: 530-537). The method specifically uses Cryptofix or lithium ion to prevent interference. U.S. Pat. No. 6,068,971 issued to Roche Diagnostics attempts to use the reaction described in U.S. Pat. No. 5,700,652 but first removes potential interfering materials. In contrast to the present invention, these patents measure change in color absorbance over time (delta A/minute). The present invention converts the measured change into an (X,Y) coordinate variable, which reduces or eliminates the experimental variations due to time, temperature, and other factors. The present invention is thus designed to be robust for use in an open environment including in locations where access to electricity is limited.

There are several patents that describe saliva collection devices for diagnostic testing. Examples of these include U.S. Pat. No. 6,372,513, which describes a glass fiber pad with salt on it known to break down the mucous in saliva, and U.S. Pat. No. 5,922,614, which comprises a foam Q-Tip, whose sleeve squeezes the foam material to release the saliva sample.

U.S. Pat. No. 6,057,139 issued to McNeil-PPC, Inc. seeks to provide lactase (beta-galactosidase) tablets for consumption. In order to provide a stabilized product, it employs microcrystalline cellulose. The lubricants are used for pressing the active ingredient into a retained pill form. The patent has not prevented other manufacturers from using microcrystalline cellulose in their own lactase products. For example, Safeway commercializes its own lactase tablets, the container for which includes a disclaimer stating that the product is not manufactured or distributed by McNeil.

### REFERENCES CITED

U.S. Patent Nos. cited are U.S. Pat. Nos. 4,649,123; 4,812,400; 5,300,439; 5,700,652; 5,766,870; 5,992,614; 6,057,139; 6,068,971; and 6,372,513.

### OTHER PUBLICATIONS

1. Cohn M, Monod J. 1951 [Purification and properties of the beta-galactosidase (lactase) of Escherichia coli]. (Article in French). Biochim Biophys Acta. 7:153-174.
2. Monod J, Cohen-Bazire G, Cohn M. 1951. [The biosynthesis of beta-galactosidase (lactase) in Escherichia coli; the specificity of induction.] (Article in French). Biochim Biophys Acta. 7:585-599.
3. Reithel F J, Kim J C. 1960. Studies on the beta-galactosidase isolated from Escherichia coli ML 308.1. The effect of some ions on enzymic activity. Arch Biochem Biophys. 90:271-277.
4. Lederberg J. 1950. The beta-d-galactosidase of Escherichia coli, strain K-12. J. Bacteriol. 60:381-392.
5. Kuby, S. A. and Lardy, H. A. 1953. Purification and kinetics of D-galactosidase from Escherichia coli, strain K-12. J. Am. Chem. Soc. 75:890-896.
6. Becker V E, Evans H J. 1969. The influence of monovalent cations and hydrostatic pressure on beta-galactosidase activity. Biochim Biophys Acta. 191:95-104.
7. Hill J A, Huber R E. 1971. Effects of various concentrations of Na⁺ and Mg²⁺ on the activity of beta-galactosidase. Biochim Biophys Acta. 250:530-537.
8. Kay G, Lilly M D, Sharp A K, Wilson R J. 1968. Preparation and use of porous sheets with enzyme action. Nature 217:641-642.
9. Sharp K, Kay G, Lilly M D. 1969. The kinetics of beta-galactosidase attached to porous cellulose sheets. Biotechnol Bioeng. 11:363-380.
10. Lilly, M. 1971 Stability of Immobilized beta-Galactosidase on Prolonged Storage, Biotechnol Bioeng 13:589.
11. Brena B M, Ryden L G, Porath J. 1994. Immobilization of beta-galactosidase on metal-chelate-substituted gels. Biotechnol Appl Biochem. 19:217-231.

### BRIEF SUMMARY OF THE INVENTION

The present invention provides both systems and methods for measuring saliva sodium concentration. Apparatus according to the present invention comprise a non-toxic saliva sample collector and a container integral to, attached to, or detached from the sample collector, which stores a chromatography buffer, typically in a chromatography chamber. The chromatography buffer is released from the chromatography chamber, typically by a physical action by the patient, for contact with the saliva sample and/or controls, either before, during, or after collection of the saliva in the collector. The buffer—which is used to dilute the saliva sample to ensure proper chromatographic migration of the saliva sample—carries the sodium in the saliva sample into a solid phase chromatography medium which typically is in the solid phase and contains an immobilized enzyme, such as beta-galactosidase, which can react with the sample to produce a detectable signal representative of the presence of sodium. Usually, the buffer, the solid phase chromatography medium, or both contains a label for the enzyme, such as a calorimetric substrate. As the buffer carries the saliva sample along the chromatography medium, sodium in the sample reacts or binds with the enzyme or label located on the medium, thereby generating a visible color or other change. Lateral flow or other transport technology enables this colorimetric reaction to continue to advance along the chromatography medium until the sodium has been depleted from the saliva sample. Sodium concentration in the sample is thus proportional to the distance the reaction front advances across the chromatography medium. The systems may include controls, which serve to refine the quantitation of saliva sodium concentration.

The saliva sample collector may have any form suitable for absorbing or otherwise collecting the required saliva and subsequently delivering a preselected (dosed) amount of the saliva together with the buffer to the chromatography medium. Usually, the sample collector will permit oral collection of saliva, such as via sucking or licking. Exemplary sample collectors include non-toxic, interference free (non-analyte participating) materials such as bite-size sponges, pads, swabs, and filter paper. Typically, the sample collector will also include a mechanism for delivering or combining a measured or calibrated amount of the collected saliva to the buffer. Such measurement or calibration may be achieved by an expanding or non-expanding medium that absorbs only a predetermined (dosed) amount of saliva, or by a physical collection device that expresses or wrings from the sample collector a predetermined (dosed) amount of saliva. The system may further provide visual indications that the requisite amount of saliva has been collected and/or delivered.

The sample collector may comprise one or more containers. Exemplary containers include tube-like devices made of plastic. The container will usually hold the chromatography chamber, which typically holds one or more doses of the buffer. The buffer is initially separated from the saliva and/or controls, preferably via foil or other physical barriers. Penetration of the barrier initiates the measurement procedure and releases the chromatography buffer from the chromatography chamber for contact with the dosed saliva sample and/or various controls.

The systems of the present invention will usually comprise an integrated device which includes the sample collector, the container, the chromatography chamber, the chromatography medium with active enzyme, the colorimetric substrate, and the controls. Alternatively, the container storing the chromatography chamber and buffer may be selectively detached from the other measurement components. For example, in an integrated element, a post-it-pen-type device may hold all measurement components. The patient or user may apply saliva to the sample collector, and then initiate the chromatographic reaction by mechanically turning a dial or pressing a button or placing or replacing a cap, whereby such action releases the buffer from the chamber for contact with the dosed sample and, potentially, various controls. In a distributed element, a device comprising the sample collector, the chromatography medium with active enzyme, and certain controls, among other components, is detached from the chromatography chamber and buffer. The patient or user may apply saliva to the sample collector and then selectively penetrate the chromatography chamber storing the buffer with the sample collectors or some other device, thereby initiating the chromatographic reaction.

The chromatography buffer will carry the sodium ions in the sample into the solid phase chromatography medium, which contains an immobilized enzyme such as beta-galactosidase. A colorimetric substrate for the enzyme may be placed within the chromatography buffer or the solid phase chromatography medium, based on chromophoric development, ease of color distinction, chemical stability, temperature stability, cost, and other factors. As the buffer carries the saliva sample along the chromatography medium, sodium in the sample binds with the enzyme located on the medium, thereby generating a visible color change. Exemplary chromatography mediums include Whatman paper, silica gel on a nitrocellulose backing, agarose gel, or other filer paper. Exemplary buffers include various sodium-free aqueous components such as buffered DDI water or saliva matrix. Exemplary substrates may include, among others, X-Gal and ONPG, which respectively produce blue and yellow colors upon reaction; selection will be based upon a number of factors, including color distinction, chemical stability, temperature stability, cost, and other factors.

The chromatography buffer migrates across the entire chromatography medium, carrying the depleted sample with it. When the sodium ions have been depleted from the sample, the enzyme on the solid phase chromatography medium, and the associated colorimetric reaction, will no longer be activated.

A number of different stabilizing additives, such as sucrose or microcrystalline cellulose, may be used to preserve the enzyme activity. Beta-galactosidase is found in food processing applications, including in the treatment of milk. Stability may be approached by immobilizing an enzyme to a solid phase support (Kay et al., 1968). The kinetics of immobilized beta-glactosidase have also been studied using Whatman paper as the support (Sharp et al., 1969). The long-term stability of immobilized beta-galactosidase has been studied and described (Lilly, 1971).

Means for measuring saliva sodium concentration via a chromatography reaction rely upon measuring the distance along the length of the chromatography medium required to deplete the saliva sample of sodium ions. The measurement system includes physical indicators which denote distance and which correspond quantitatively or qualitatively to sodium concentration levels.

In order to equate sodium concentration to numerical values of sodium molarity, one or more controls, or parallel channels containing dosed sodium solutions, may be run simultaneously. If standard concentrations of Na+ ions are run concurrently with saliva samples, the buffer matrix must produce a result that correlates with actual saliva.

In order to facilitate depletion, the beta-galactosidase concentration in the solid phase chromatography medium need not be constant. The solid phase nearest the sample collector may contain higher concentrations of the enzyme, thereby facilitating rapid depletion of a portion of the sodium early in the chromatography procedure. Similarly, the solid phase nearest the end of the medium may contain lower concentrations of the enzyme, thereby stretching the physical distance that distinguishes medically interesting molar concentrations of sodium.

The present invention further provides methods for determining a level of dehydration in a patient based upon measured saliva sodium concentration.

The present invention further provides methods for rehydrating patients. The methods rely on determining a level of dehydration in the patient, where such determination is based upon saliva sodium concentration, and preparing a rehydration fluid by combining an amount of sodium with an aqueous agent, among other ingredients. Particularly, the methods provide that the amount of sodium to be combined is selected based on the determined level of dehydration. The level of patient dehydration is based upon measured saliva sodium concentration as described above. The rehydration fluid is then prepared by mechanically releasing a calibrated amount of sodium into the aqueous component, typically in a drinking vessel as described in U.S. Provisional Patent Application No. 60/603,949, “System and Method for Controlling the Fluid and Electrolyte State of a Patient,” the full disclosure of which has been previously incorporated by reference.

The present invention still further provides systems or kits including both the measurement devices described herein and the drinking systems described in application No. 60/603,949. A patient can use the measurement device to determine a level of rehydration and, based on that level, calibrate the drinking device to combine the proper amount of sodium and/or other electrolyte(s) to provide a rehydration fluid intended to specifically address the individual level of dehydration. The measurement device can be attached or otherwise coupled to the drinking device or may be detached.

The present invention has certain objects. That is, the present invention provides solutions to problems existing in the prior art. It is an object of the present invention to provide a system for measuring saliva sodium concentration that is rapid, non-invasive, disposable, and low-cost, thereby enabling individuals to monitor fluid and electrolyte levels in an outpatient setting. Another object of the present invention is to diagnose various fluid and electrolyte disorders in an outpatient setting, thereby enabling patients to make informed healthcare decisions. Another object of the present invention is to provide a method for titrating fluid and electrolyte delivery based on actual fluid and electrolyte replacement needs, thus combining oral delivery therapies for administering fluid and electrolytes with monitoring technologies so as to effect tight control over the fluid and electrolyte level of a patient. The optimal rehydration solution varies widely from patient to patient, and inter-patient over time, based on a number of different factors. The system of the present invention can measure sodium replacement requirements, enabling the dosing of a rehydration solution based on the unique biometric needs of the patient.

Various embodiments of the present invention have advantages, including one or more of the following: (a) enabling patients to diagnose various fluid and electrolyte disorders in an outpatient setting; (b) improving the direct or indirect control that may be exercised over the fluid and electrolyte levels of a patient; (c) quickly enabling the delivery of the required amount of sodium to a patient before hypernatremia, hyponatremia, volume depletion or edema develop or become life threatening; (d) overcoming the deficiencies of relying on “best guess” estimates of fluid and electrolyte replacement requirements, either or both of which are often under- or overestimated by patients; (e) reducing the number and severity of medical complications, thereby increasing patient safety and lowering health care costs due to better control of patient fluid and electrolyte levels.

Various embodiments of the present invention have certain features. In one embodiment of the present invention, measured patient saliva sodium concentration data is used to inform healthcare decision making. In another embodiment, the measurement system wirelessly or electronically transmits measured patient saliva sodium concentration data to a diagnostic or monitoring device either integral to or separate from the system of the present invention, which in turn generates a set of commands for a fluid and/or electrolyte delivery system. For example, the system of the present invention may be built into the mouthpiece of a container for drinking. A patient places his lips on the mouthpiece of the container, such action generates a saliva sodium concentration reading, such reading is transmitted to a receiving system which, based on the data transmitted from the system of the present invention, sends a series of commands to the delivery system, which then releases a proportional amount of beneficial agents contained in a retention pocket integral or attached to the container for drinking. In this embodiment, the control strategy of the system is preferably microprocessor based and/or implemented using dedicated electronics. Such a control strategy would enable the delivery system to generate patient data, such as fluid and/or electrolyte trends, which data may be used to further refine future calculations of fluid and/or electrolyte replacement needs.

By measuring saliva sodium concentration, and adjusting rehydration therapy based on this data, individuals can reduce the long-term threats associated with renal and cardiovascular complications. The systems and methods of the present invention constitute a reliable saliva sodium concentration measurement system that permits enhanced, tight control of patient fluid and electrolyte levels, among other medical conditions.

Additional objects, advantages, and embodiments of the invention will be realized by the method and system described in the written description and claims hereof, as well as from the appended drawings. It is to be understood that both the foregoing general description and the following detailed description are exemplary and are intended to provide further explanation of the invention claimed.

### BRIEF DESCRIPTION OF THE DRAWINGS

FIG. 1 illustrates an unassembled view of one embodiment of the monitor.

FIG. 2 illustrates the basic color development principle.

FIG. 3 illustrates the profile of sodium depletion across the test strip.

FIG. 4 illustrates the alternative distributions of beta-galactosidase.

FIG. 5 illustrates the expected results as a function of sodium concentration.

FIG. 6 illustrates the correlation between serum sodium concentration and serum osmolality.

### DETAILED DESCRIPTION OF THE INVENTION

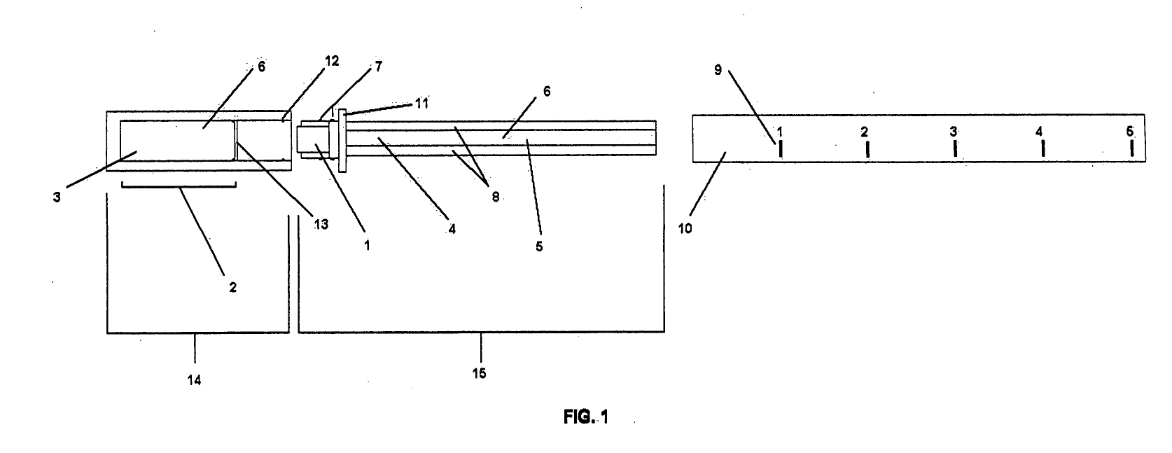

FIG. 1 illustrates one possible embodiment, wherein the cap 14 is comprised of a chromatography chamber 2, which contains the chromatic buffer 3 (which may or may not contain a calorimetric substrate for the enzyme 6), and a foil vapor barrier 13. Additionally, the cap has protrusions 12, which will interface with protrusions 7 on the plastic collar 11.

The diagnostic mechanism 15 is comprised of a sample collector 1, which is mounted on the plastic collar 11. Also attached to the plastic collar is the solid state chromatography medium 4 with embedded enzyme 5. The solid state chromatography medium may or may not contain a calorimetric substrate for the enzyme 6. Additionally, one or more controls 8 may also be located in this mechanism.

The body 10 is made of a transparent material with low or zero vapor permeability. Printed or molded into this body 10 are physical markers that correlate to sodium concentration 9.

As shipped to the user, the cap 14 is attached to the plastic collar 11, with the cap protrusions 12, interfacing with the first set of plastic collar protrusions 7. In this position, the cap is securely attached, but the plastic collar 11 has not punctured the foil vapor barrier 13. The body 10 is permanently attached to the plastic collar, encompassing the solid state chromatography medium 4, embedded enzymes 5, and controls 8. The user removes the cap 14, inserts the sample collector 1 into his/her mouth transferring a quantity of the user's saliva onto the sample collector 1. The cap 14 is then reattached, with the user pushing the cap far enough for the cap protrusion 12 to interface with the second set of plastic collar protrusions 7. This action is accompanied by tactile and audible feedback signaling that the plastic collar 11 has punctured the foil vapor barrier 13, releasing the chromatography buffer 3. The saliva buffer 3, solid phase chromatography medium 4 with embedded enzyme 5, and calorimetric substrate 6 result in a length of color change along the chromatography medium 4 proportional to the amount of sodium contained in the saliva sample. This result can be viewed through the transparent wall of the body 10, and compared to the physical markers 9 located on the walls of the body 10.

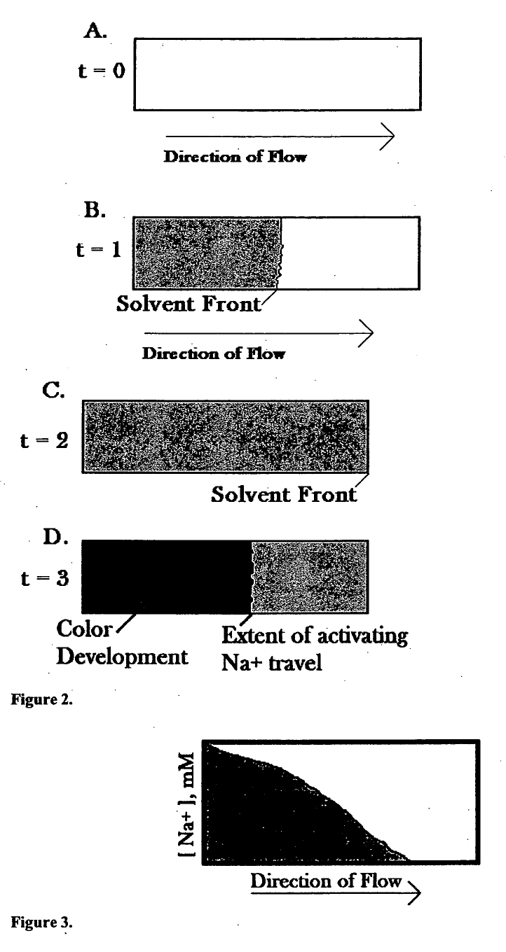

FIG. 2 illustrates the basic color development principle.

A. At time t=0, the test strip has no color development. The sample will be applied to the left edge of the strip, and will migrate toward the right.

B. At some time t=1 after application of the sample, the combined sample and buffer will migrate across the test strip forming a solvent front. The indicated solvent front may not be exactly perpendicular to the direction of flow. The binding of sodium (Na+) in the sample with beta-galactosidase will initiate the colorometric reaction. The wetted strip surface may attain some level of background color as indicated by the rose color.

C. At some time t=2, the solvent front will reach the physical limit of the strip, halting further migration. At this time, Na+ migration due to chromatographic action will cease. Diffusion may occur, but should not be apparent within the time scale of the reaction.

D. By some time t=3, the chromogenic substrate of beta-galactosidase will react on that portion of the strip with Na+ in the sample. Because the Na+ of the sample will be depleted by binding to beta-galactosidase during travel, a point will be reached after which all Na+ detectable by enzymatic activity is bound. The strip to the right of this point will exhibit little or no color change. The indicated extent of activating Na+ travel may not be exactly perpendicular to the direction of flow.

FIG. 3 illustrates the profile of sodium depletion across the test strip. As the buffer carries the sample across the beta-galactosidase-impregnated test strip, Na+ ions will be retained by the enzyme in passing until such a point in the migration beyond which Na+ can no longer be detected. The shape of the decay curve may be influenced by several factors such as enzyme concentration.

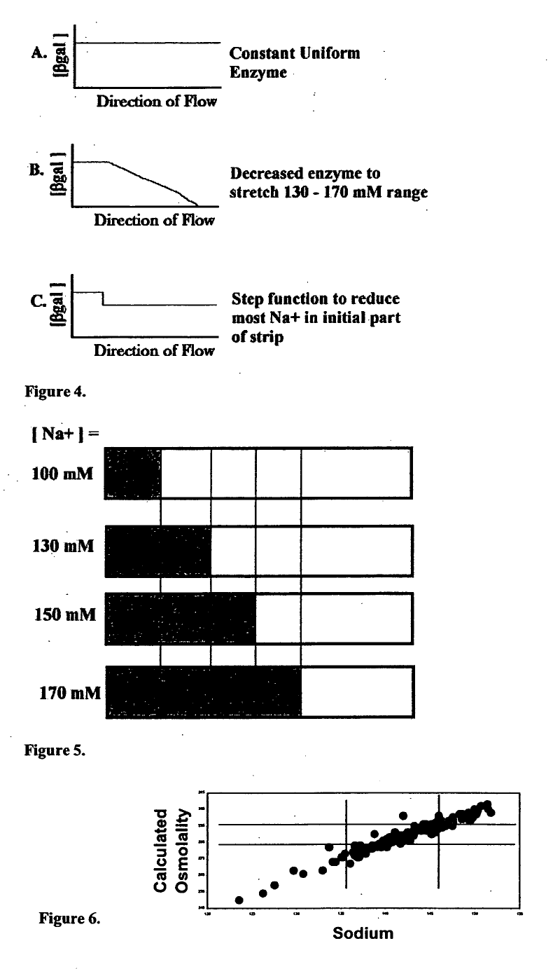

FIG. 4 illustrates the alternative distributions of beta-galactosidase.

A. The enzyme may be distributed evenly at constant concentration on the test strip for ease of application and quality control.

B. Alternatively, the enzyme may be applied with a linear, parabolic, exponential, or other decay function. This will provide the most effective Na+ binding and removal in the initial phase of chromatography. The later portion will thus require a longer travel distance to remove the last amount of Na+, better distinguishing in the 130-170 mM range.

C. The enzyme may be applied in a step function as a simpler application than nonlinear gradients. This option retains the enhanced removal of most Na+ in early stages of the assay. This portion may or may not be made visible to the user.

FIG. 5 illustrates the expected results as a function of sodium concentration. As the sample concentration increases from 100 to 170 mM Na+, the distance traveled before the Na+ is depleted by the enzyme will increase. The assay may be adjusted to ensure that samples do not reach full travel under normal physiological conditions. If controls are co-run with donor samples, the distance traveled must be equal for the same Na⁺ concentration in saliva as in the standard matrix buffer.

What is claimed is:

1. A measurement system comprising:
a sample collector adapted to collect a saliva sample;
a measurement device comprising a chromatography chamber and a chromatography buffer;
a solid phase chromatography medium, said medium having a labeling system which produces a visible label in the presence of sodium, and
wherein the sample collector is configured to deliver collected sample to the chromatography medium.

2. A measurement system as in claim 1, wherein the sample collector comprises an absorptive material selected from the group consisting of a bite-size sponge, a pad, a swab, or filter paper.

3. A measurement system as in claim 1, wherein the sample collector absorbs patient saliva as a result of a patient-initiated action such as licking, sucking, or applying saliva to the sample collector.

4. A measurement system as in claim 1, wherein the sample collector absorbs a dosed amount of saliva, thereby creating a dosed saliva sample.

5. A measurement system as in claim 4, wherein said dosed amount of saliva is subsequently expressed from the sample collector by squeezing or plunging or wringing or some other mechanical means.

6. A measurement system as in claim 1, wherein the sample collector absorbs a non-dosed amount of saliva.

7. A measurement system as in claim 6, wherein a portion of said non-dosed amount of saliva is subsequently removed by a secondary device, which squeezes or plunges or wrings a dosed amount of saliva from the sample collector, thereby creating a dosed saliva sample.

8. A measurement system as in claim 1, wherein the user is alerted visually when the sample collector has collected the minimum amount of saliva necessary to complete the measurement.

9. A measurement system as in claim 1, wherein the container is most often, but not necessarily, made of plastic, glass, aluminum, stainless steel, rubber, or some combination thereof.

10. A measurement system as in claim 1, wherein the container comprises a cup, tube, bladder, or box.

11. A measurement system as in claim 1, wherein the container is formed integrally with the sample collector.

12. A measurement system as in claim 1, wherein the container is reasonably attached to the sample collector.

13. A measurement system as in claim 1, wherein the container is reasonably detached from the sample collector.

14. A measurement system as in claim 1, wherein the container holds one or more chromatography chambers.

15. A measurement system as in claim 1, wherein the chromatography chamber(s) store a single dose, or a plurality of doses, of a chromatography buffer.

16. A measurement system as in claim 1, wherein a dose of the chromatography buffer is released from the chromatography chamber(s) for contact with the dosed saliva sample.

17. A measurement system as in claim 1, wherein a dose, or a plurality of doses, of the chromatography buffer are released from the chromatography chamber(s) for contact with one, or multiple, controls.

18. A measurement system as in claim 1, wherein the buffer is separated from the dosed saliva sample via a foil cover or other mechanical barrier.

19. A measurement system as in claim 16, wherein the release mechanism comprises piercing a foil cover or other pierceable member of the chromatography chamber; mechanically or electronically moving or removing a physical barrier between the buffer and the dosed saliva sample or controls; turning a dial; transforming the state of the physical barrier between the buffer and the dosed saliva sample or controls, for example, from solid to liquid; or pushing or pulling a lever or button.

20. A measurement system as in claim 1, wherein the chromatography buffer comprises a sodium-free, aqueous component, such as buffered DDI water or saliva matrix.

21. A measurement system as in claim 1, wherein the chromatography buffer includes a colorimetric substrate for beta-galactosidase.

22. A measurement system as in claim 1, wherein the chromatography buffer includes stabilizers, said stabilizers serving to preserve the enzymatic activity, and/or stabilize the buffer, and/or preserve the calorimetric substrate.

23. A measurement system as in claim 22, wherein the stabilizers comprise microcrystalline cellulose.

24. A measurement system as in claim 22, wherein the stabilizers comprise glycerol, said glycerol serving to stabilize the buffer and/or to prevent freezing.

25. A measurement system as in claim 1, wherein a solid phase chromatography medium contains immobilized beta-galactosidase.

26. A measurement system as in claim 1, wherein the solid phase chromatography medium contains constant or varying concentrations of the immobilized enzyme.

27. A measurement system as in claim 1, wherein the solid phase chromatography medium comprises Whatman paper, silica gel on a nitrocellulose backing, agarose gel, or other filter paper.

28. A measurement system as in claim 1, wherein the chromatography medium includes a colorimetric substrate for beta-galactosidase.

29. A measurement system as in claim 21, wherein the colorimetric substrate comprises X-Gal, ONPG, or other calorimetric substrates.

30. A measurement system as in claim 1, wherein the means for measuring saliva sodium concentration via a chromatographic reaction comprise combining a dosed saliva sample with a chromatography buffer, said combination providing the chromatographic impetus along the length of a solid phase chromatography medium.

31. A measurement system as in claim 1, wherein the means for measuring saliva sodium concentration via a chromatographic reaction comprise the binding of sodium ions in the dosed saliva sample with beta-galactosidase, said binding producing a colorimetric reaction via a colorimetric substrate for the enzyme.

32. A measurement system as in claim 1, wherein the means for measuring saliva sodium concentration via a chromatographic reaction comprise observing the distance along the length of the chromatography medium required to deplete the dosed saliva sample of sodium ions, said distance measured by physical markers, said markers calibrated according to the sodium concentration of the dosed saliva sample.

33. A measurement system as in claim 32, wherein the calibration of saliva sodium concentration may take a numerical form corresponding to standard saliva or plasma sodium concentrations (e.g. sodium concentration of 135 mEq/L), a numerical form which does not correspond to standard sodium concentrations (e.g. level 1, 2, 3), a qualitative form (e.g. high, normal, low), or a combination thereof.

34. The measurement system of claim 1, wherein the means for measuring saliva sodium concentration via a chromatographic reaction comprise one or more controls.

35. The measurement system of claim 1, wherein patient saliva sodium concentration data is wirelessly transmitted to the receiving system of a diagnostic, monitoring, or data processing device.

36. The measurement system of claim 35, wherein said wirelessly transmitted patient data is electronically processed and used to generate commands for a fluid and/or electrolyte delivery system.

37. A method for determining a level of dehydration in the patient, wherein the level of dehydration is based upon measured saliva sodium concentration.

38. A method for rehydrating a patient, said method comprising:
determining a level of dehydration in the patient; and
preparing a rehydration fluid by combining an amount of sodium with an aqueous component, wherein the amount of sodium is selected based on the determined level of dehydration.

39. A method as in claim 38, wherein the level of dehydration is based upon measured saliva sodium concentration.

40. A method as in claim 38, wherein the rehydration fluid is prepared by mechanically releasing a calibrated amount of sodium into an aqueous component in a drinking vessel.

41. A rehydration system comprising:
a measurement device which analyzes a patient sample to determine a level of rehydration; and
a drinking device which combines an aqueous component and an amount of an electrolyte selected based on the measured level of dehydration.
